(19) United States
(12) Patent Application Publication
(10) Pub. No.: US 2009/0274592 A1
(43) Pub. Date: Nov. 5, 2009

(54) PLASMA-BASED AIR PURIFICATION DEVICE INCLUDING CARBON PRE-FILTER AND/OR SELF-CLEANING ELECTRODES

(75) Inventor: Vance BERGERON, Francheville (FR)

Correspondence Address: Beyer Law Group LLP, P.O. BOX 1687, Cupertino, CA, 95015-1687, US

(73) Assignee: AirlnSpace B.V., Delft (NL)

(21) Appl. No.: 12/428,310

(22) Filed: Apr. 22, 2009

Related U.S. Application Data
(60) Provisional application No. 61/049,668, filed on May 1, 2008.

(51) Int. Cl. B03C 3/38; B03C 3/01; B03C 3/017; A61L 9/22; B03C 3/41; A61L 9/00

(52) U.S. Cl. 422/186.29; 422/186.04; 96/57; 96/95

(57) ABSTRACT

In one aspect of the invention, a plasma reactor is arranged to treat aerosol particulates in a fluid stream passing through the reactor. The plasma reactor includes a plasma chamber having a self-cleaning electrode. The self-cleaning electrode is configured to clean various residues from the electrode without need to open or otherwise service the unit. In another aspect, the invention comprises a carbon-based pre-filter arranged to filter in flowing air to reduce the amount of silicone-based contaminant in the air flow before the air reaches the ionization chamber.

### CROSS REFERENCE TO RELATED APPLICATION

This application claims priority to the U.S. Provisional Patent Application No. 61/049,668, filed on May 1, 2008, entitled “Plasma-Based Air Purification Device Including Carbon Pre-Filter and/or Self-Cleaning Electrodes”, which is hereby incorporated herein by reference.

### BACKGROUND OF THE INVENTION

The present invention generally relates to air cleaning and purification devices. More particularly, the invention relates to self-cleaning electrode arrangements suitable for use in such devices including plasma based air cleaning and purification devices.

There are currently a wide range of technologies that are used to purify and/or filter air. One such technology is the ion enhanced electrostatic filter. An ion enhanced electrostatic filter contemplates placing an ion source in front of the electrostatic filter to impart an electric charge to some of the particulates carried by air passing through the filter. Commonly, the ion source uses an electrode to impart an electrical charge to particles flowing through a fluid stream (e.g., air). After the particles are charged, they are passed through an active electrostatic filter where they can be removed from the fluid stream. The charges imparted to the particulates by the ionizer tend to help their collection within the dielectric active electrostatic filter. Thus, the presence of the ionizer imparts a charge sufficient to cause the particulates within the air stream to adhere to a dielectric filter as they exit an ionizer and pass through the filter.

U.S. Pat. No. 5,474,600, which is owned by the assignee of the present patent, discloses an apparatus for the biological purification and filtration of air. Generally, the '600 patent discloses a system which utilizes a course electrostatic filter 1, a cylindrical or polygonal ionizer 5 and a fine electrostatic filter 10 that are all arranged in series. In some of the described embodiments, a pair of ionizers that impart opposite charges are arranged in series between the course and fine electrostatic filters. The system is arranged to inactivate (i.e. kill) biological objects (e.g., microorganisms and viruses) that are carried in the air stream and to filter particulates from the stream.

Another typical embodiment of such a system is diagrammatically illustrated in FIG. 1. As seen therein, the system 20 includes an electrostatic pre-filter 22, a positive plasma generator 24 that is arranged in series with a negative plasma generator 26 and a series of four electrostatic filters 28 that are arranged downstream of the negative plasma generator 26. Each D.C. plasma generator 24, 26 is composed of a plurality of cylindrical plasma cylinders (e.g., 6 cells) arranged in parallel. Each cell has a needle type ionizing electrode 27 that is surrounded by a cylindrical electrode chamber. One of the electrodes is grounded while a D.C. potential of either 4000 or 7600 volts is applied to the opposing electrode. The electrostatic filters may be formed as described in U.S. Pat. Nos. 5,474,600 or 6,805,732 and plasma generator may be formed as described in U.S. Pat. No. 5,474,600 or U.S. Pat. No. 7,198,660. All of these patents and patent applications are incorporated herein by reference.

In another type of device, the ionizer simply comprises an electrically charged wire grid. As the air stream flows through the ionizer an electrical charge is imparted to particulates flowing through the mesh. After these particles are charged, they are passed through an active electrostatic filter where they can be removed from the fluid stream.

The inventors point out that due to the extremely high voltages used with ionizers of this type, the electrodes can suffer from a build up of contamination which over time can degrade the effectiveness of the ionizers. What is needed is an approach for addressing these contamination issues. Such approaches are discussed in this in this document.

Thus, although existing electrodes work well enough, there are opportunities for improvement and continuing efforts to provide improved discharge electrodes that can meet the needs of various applications.

### SUMMARY OF THE INVENTION

In one aspect of the invention, a plasma treatment unit with an enhanced electrostatic filter is described. In such a unit, an initial stage includes a carbon-based pre-filter arranged to capture silicone and silicone based residues from an inflowing fluid stream. A next stage comprises a plasma reactor arranged to treat aerosol particulates in the fluid stream passing out of the pre-filter and through the reactor. The plasma reactor includes a plasma chamber having a self-cleaning discharge electrode that charges the particles as they pass through the chamber. The charged particles are then passed through an enhanced electrostatic filter which captures the charged particles. A porous catalyst can be added at the outflow of the plasma reactor to neutralize undesirable species contained in the air flowing from the plasma reactor prior to the filtered air being introduced into the ambient environment. For example, the catalyst can be used to neutralize ozone produced by the plasma reactor. In some embodiments, a self-cleaning discharge electrode is used to provide enhanced residue removal from the electrode without need to open or otherwise service the unit.

In some implementations the self-cleaning electrodes comprise an elongate discharge electrode (e.g., a discharge needle or wire loop or other electrode embodiment) that is arranged near a complementary counter-electrode (also referred to herein as a receptor electrode or a “receptor”) of a plasma or ionization chamber. A cleaning mechanism is arranged in the apparatus to enable cleaning the discharge electrode without opening the unit. Such a cleaner includes a cleaning surface arranged so that the cleaner and the discharge electrode can be moved into cleaning contact with each other to facilitate cleaning of the discharge electrode.

Generally, the various aspects of the invention may be used separately or in combination with one another.

### BRIEF DESCRIPTION OF THE DRAWINGS

The invention, together with further objects and advantages thereof, may best be understood by reference to the following description taken in conjunction with the accompanying drawings in which:

FIG. 1 diagrammatically illustrates an existing plasma based air purification and filtering system;

FIG. 2A diagrammatically illustrates a plasma based air purification and filtering system in accordance with an embodiment of the present invention;

FIGS. 2B and 2C diagrammatically illustrate some embodiments of a pre-filtration element for a plasma based air purification and filtering system in accordance with an embodiment of the present invention;

FIG. 3A is a diagrammatic end view of a plasma generator that is composed of a plurality of adjacent hexagonal plasma chambers arranged in parallel;

FIG. 3B is a diagrammatic end view of a plasma generator that is composed of a plurality of adjacent cylindrical plasma chambers arranged in parallel;

FIG. 3C is a diagrammatic section view of a plasma generator composed of a cylindrical plasma chamber;

FIGS. 4(a) & 4(b) diagrammatically illustrate the functionality of one embodiment of a self-cleaning discharge electrode having a cleaning collar/sheath;

FIGS. 5(a) & 5(b) diagrammatically illustrate the functionality of another embodiment of a self-cleaning discharge electrode having an alternative cleaning collar arrangement;

FIGS. 6(a)-6(d) diagrammatically illustrate a self-cleaning discharge electrode embodiment having a brush cleaner suitable for use in accordance with another aspect of the present invention;

FIGS. 7(a)-7(d) are diagrammatic views of a self-cleaning line or wire loop discharge electrode and embodiments of example cleaning elements suitable for use in cleaning the electrode of the invention;

FIGS. 8(a)-8(b) are diagrammatic illustrations of a discharge electrode and receptor plate arranged to ionize airflow material and embody discharge electrode cleaning elements in accordance with the principles of the invention;

FIGS. 9(a)-9(b) are diagrammatic illustrations of a discharge electrode and receptor plate embodiment having cleaning elements in accordance with the principles of the invention;

FIGS. 10(a)-10(c) are diagrammatic illustrations of a discharge electrode and receptor plate embodiments having cleaning elements arranged to clean the discharge electrodes an/or the receptor in accordance with the principles of the invention;

It is to be understood that, in the drawings, like reference numerals designate like structural elements. It should also be understood that the depictions in the figures are diagrammatic and not to scale.

### DETAILED DESCRIPTION OF SELECTED EMBODIMENTS

The present invention relates generally to the cleaning of electrodes used in air purification devices that can decontaminate, filter and/or purify an air flow. In particular, the present invention is applicable to plasma treatment units using enhanced electrostatic filters.

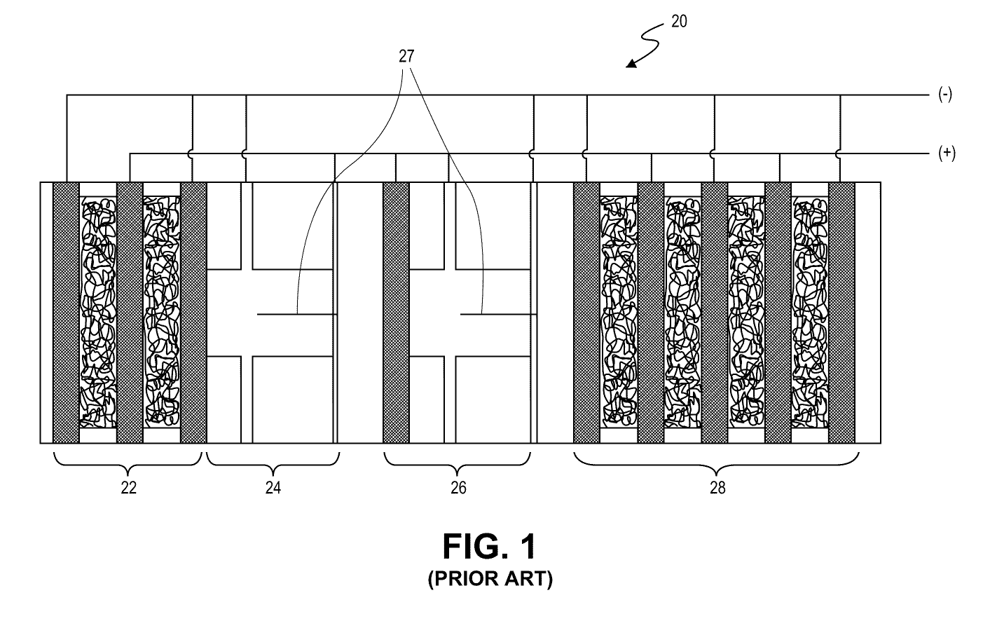

Referring, for example, to the air cleaning device described in FIG. 1 above, the patentees have discovered that, over time, the discharge (ionization) electrodes 27 of the ionizers undergo a steady accumulation of residue which deposits onto the surface of the electrode. This residue can have a number of negative effects. For example, if the residue is dielectric in nature it can have a deleterious effect on the electrodes ability to ionize particulate matter passing through the ionizer. Also, if the residue is of opposite charge of the electrode it attaches to the electrode and degrades electrode effectiveness. Another problem occurs when electrically insulative contaminant materials deposit on the discharge electrode.

Although employed in many different environments, in one particular embodiment, the devices described herein can be employed in a household air cleaning environment. In household environments, many volatile organic compounds (VOC's) are found in the air. In particular, silicone-based organic polymers can be found in the air. One typical contaminant of this type is dimethicone which can be particularly common in hairsprays and other household products. One common purpose of filtration units is to reduce the amounts of VOC's from the air. However, the inventors have discovered that over time the ionization of dimethicone and other silicone-containing VOC's leads to the production of silicon-based residues, particularly SiO₂ (also referred to as silica). Moreover, silicone and other household contaminants can also lead to the production of other electrically insulating residues. These residues are particularly troublesome for a number of reasons. For one, they readily adhere to the ionization electrodes. Additionally, over time, the build up of insulative residues (SiO2 and the like) on the ionization electrodes becomes thick enough to affect the electrical properties and ionization performance of the electrodes. Over enough time the insulating properties of the silicone can render the electrodes ineffective for their intended use. Accordingly, the inventors have determined that a means for cleaning the electrodes is important. Moreover, due to the nature of some cleaning devices it can be advantageous to clean the electrodes without removing the electrodes from the air cleaning devices.

Currently, electrode cleaning is accomplished manually by opening up the devices and then individually cleaning the electrodes. However, the inventors point out that these ionizers 24, 26 are frequently enclosed inside sealed enclosures. Thus, the internal portions of the devices may not be easily accessed. Moreover, the devices themselves may be located in difficult to reach or maintenance locations. Additionally, these enclosures and devices are put into use with the idea that very little maintenance needs to be performed on the devices. Thus, manual cleaning has the disadvantage of driving up maintenance costs and making the devices more difficult to maintain. This is particularly problematic with low cost and consumer sized units.

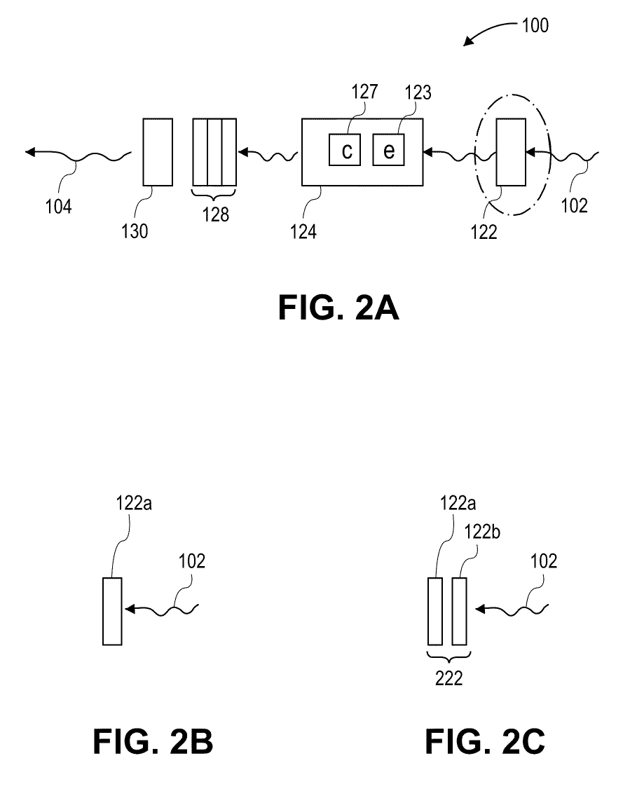

FIG. 2A schematically depicts one elementary embodiment of an air cleaning device of the invention. The depicted device 100 is a “plasma enhanced electrostatic filtration unit” also referred to herein as a plasma reactor. Such devices are to be distinguished from electrostatic precipitator units which are a different technology. In FIG. 2A, an example device depicts an inflowing air stream 102 passing through the various elements of the reactor 100 to produce a cleaned outflowing air stream 104. In one embodiment, the reactor 100 includes a pre-filter 122 which can remove the gross particulate matter from the inflowing air stream 102. The device further includes, a plasma generator 124 (which can comprise one or many such generators and can comprise both positive and negative plasma generators) including an ionization (or discharge) electrode 123, at least one counter-electrode 125, an ionization electrode cleaner 127, one or a series of enhanced (or active) electrostatic filters 128 and can include a catalyst 130 that operates as a catalytic converter enabling the removal of ozone and other active species from the cleaned air stream 104. These components are all arranged in series so that a gaseous fluid stream 102 (e.g. air) enters the pre-filter 122 and sequentially passes through the plasma generator 124, the electrostatic filter 128 and the catalyst 130 to exit as a cleaned air stream 104.

In one particular embodiment, the pre-filter 122 is configured as a common filter element such as a HEPA (high efficiency particulate air) filter. Alternatively, a low efficiency filter can be used. Such low efficiency filters can provide particular utility in high throughput applications where large air volumes must be moved through the system quickly. The inventors have discovered that silicone-based materials and their residues can degrade system efficiencies (such as well described in later paragraphs). Accordingly, methods for removing silicone compounds and their residues from the system or for preventing them from entering the system are thought to be advantageous.

The inventors have discovered that by implementing a carbon filter element in the pre-filter 122 stage, the amount of silicone based materials can be substantially reduced. As depicted in FIG. 2B, the inventors contemplate that a simple carbon filter element 122a can be introduced in the pre-filter stage (as defined by the alternating dotted and dashed circle) to enhance the filtration and/or deactivation of silicone-based contaminants. As previously discussed the filters can be, for example, carbon containing low efficiency filters, HEPA filters, ULPA filters, SULPA filters, and other filters can be employed to treat the incoming air flow 102. In another embodiment, as depicted in FIG. 2C, the inventors contemplate that a multi-stage filter element 222 can be used. Such filter elements 222 can include two or more specialized filter elements arranged to filter the air flow 102. In the depicted embodiment, a standard (i.e., non-carbon) air filter 122b can operate to filter the air flow 102 followed by a carbon filter element 122a. Such an arrangement can enhance the filtration and/or deactivation of silicone-based contaminants. Additionally, the order of the filters 122a, 122b can be altered as circumstances and design considerations dictate. Either of the filters can be, for example, low efficiency filters, HEPA filters, ULPA filters, SULPA filters, and other filters can be employed to treat the incoming air flow 102.

Of particular note, the plasma enhanced electrostatic filtration units of the type described herein include at least a first stage comprising a plasma chamber 124 for generating ionized plasma as the air 102 passes into the chamber 124. The plasma chamber 124 includes a discharge or ionizing electrode 123 arranged in operable proximity to the counter-electrode 125. Many examples of such arrangements are described herein. For example, an elongate needle can operate as a discharge electrode 124 that is positioned inside a cylindrical counter-electrode 125 arranged so that the air flow passes through the counter-electrode 125 and the associated ionization field between the electrode and counter electrode. This ionizes particulate matter in the chamber. The inventors contemplate many alternative approaches such as discharge electrodes arranged between counter-electrode plates and so on. In particular, the inventors point out that the discharge electrodes can be needles, or wires, or other narrowly dimensioned structures as well as plates. Once the air flow is ionized it passes to an enhanced electrostatic filter element 128. The enhanced electrostatic filter element 128 is constructed of a porous filter element arranged between oppositely charged elements. In particular, in one particularly advantageous embodiment the filter is constructed of a porous dielectric medium. In low air volume applications the filter can be a high efficiency dielectric filter element (i.e., the porosities are very small enabling substantial filtration of even very small particulates). However, for higher volume applications a low efficiency dielectric filter element can be used (having larger porosities therefore enabling higher air throughput). Importantly, the inventors point out that the oppositely charged elements on opposing sides of the porous dielectric medium orient the dipoles of the dielectric material causing an induced electrical field in the porous dielectric medium. This induced electrical field enables extremely high filtration in ionized material such that even low efficiency filters have extraordinarily high filtration efficiency without the drawback of low volume air flow. This filtered air is then exhausted out of the device or optionally through a catalyst and then out of the device.

These plasma reactors, as described above (e.g., the apparatus illustrated in FIGS. 1 & 2) are different than traditional ion enhanced electrostatic filters in part because its ionizers (plasma generators) provide significantly higher ionization levels than traditional ion enhanced electrostatic filters. By way of example, traditional ion enhanced electrostatic precipitation filters may utilize current densities on the order of 2 μA/cm² (micro-Amperes per square centimeter) and generate a composite (average) electron density on the order of 10¹² electrons/m³. In contrast, the plasma generating chambers described above may utilize current densities on the order of 3.5 μA/cm² and generate a composite electron density on the order of 10¹³ electrons/m³, which improves the electron concentration (and thus the ion concentration) by about an order of magnitude. Such devices have been observed to have significant advantages over traditional air purifying and/or filtering systems. However, the higher densities also can result in a higher incidence of electrode contamination. Although the principle of the invention are well applied to such high electrical field embodiments, the may also be applied to systems with lower electron and current densities.

### The Plasma Generators

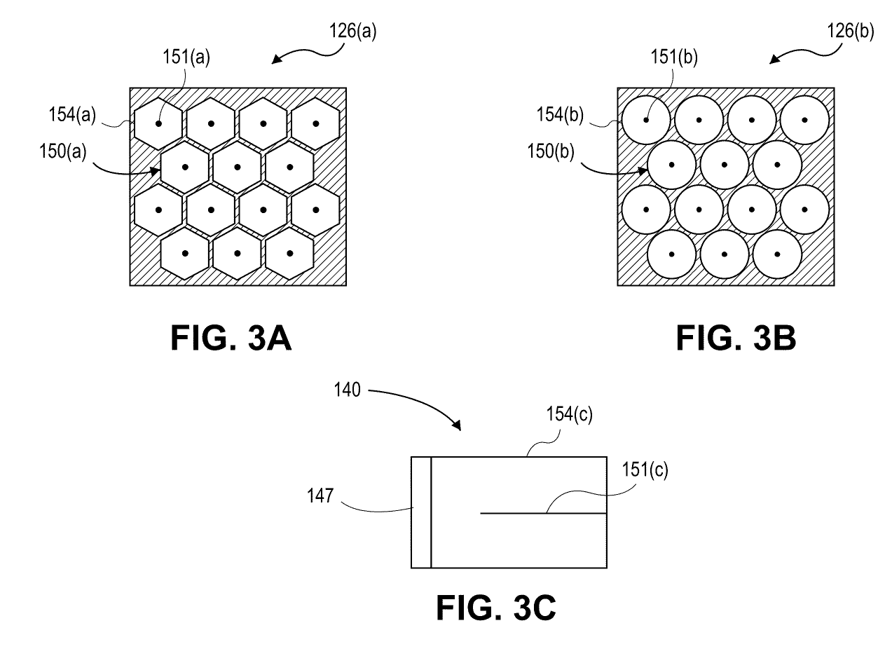

The self-cleaning electrodes of the present invention can be located within ionizing chambers (also referred to as plasma generating chambers) of the reactor or arranged in other configurations. Again, referring to FIGS. 1 & 2A, the plasma generators (e.g., 24, 26, 124) are arranged to generate a charged cold plasma. Such plasmas can be positive, negative, or both depending on the system. In the embodiment shown, the plasma generator is composed of a plurality of adjacent plasma chambers that are arranged in parallel, as illustrated in FIGS. 3A, 3B, & 3C. Suitable plasma generator arrangements are described in the aforementioned '600 patent and also the aforementioned U.S. Pat. No. 7,049,660, which are both incorporated herein by reference. For example, FIG. 3C is a cross-section view of a plasma generator chamber 140. Each chamber 140 includes a needle-type discharge electrode 151(c), chamber walls 154(c) and a receptor electrode 147. The chamber walls 154(c) can also configured to operate as a receptor. When the pre-filter is an active electrostatic pre-filter, the receptor electrode 147 may optionally also be used as one of the electrodes in the pre-filter.

The diagrammatic illustration of FIG. 3C shows a single chamber plasma generator. However, in many implementations it is desirable to provide a plurality of parallel plasma chambers for each plasma generator. For example, FIG. 3A shows a plasma generator with 12 adjacent and parallel plasma chambers. Each plasma chamber 150(a) having a hexagonal cross section with co-axial needle-type discharge electrode 151(a). FIG. 3B diagrammatically illustrates an alternative plasma generator composed of 12 adjacent plasma chambers 150(b) arranged in parallel, with each plasma chamber having a circular cross section with co-axial needle type discharge electrode 151(b). The inventors point out that the cross sectional shape of the chambers may be any appropriate shape (e.g., elliptical or polygon shaped). These chambers extend generally in the direction of the airflow with the discharge electrodes extending substantially parallel to the airflow and generally co-axially with the chamber walls. These types of plasma chambers are generally referred to herein as co-axial plasma chambers. The inventors specifically point out that the principles of the invention are not confined to needle discharge electrodes or co-axial plasma chambers. They can be applied to grid type electrodes and to other electrode types.

In one particular implementation (such as shown in FIGS. 3A & 3C), the chamber walls 154 are cylindrical and have an internal diameter in the range of 0.5 to 10 cm (as for example 5 cm). The discharge electrodes 151 are positioned co-axially with the chambers. In another particular implementation (e.g., FIG. 3B), the chamber walls are hexagonal and have minimum chamber widths in the range of 0.5 to 10 cm (as for example 5 cm).

Although, the described co-axial plasma chambers work very well and can be constructed at a relatively modest cost, it should be appreciated that a variety of other ion generating technologies may be used to create the desired plasmas or ionization zones. For example, grid electrodes could be employed as can a plurality of discharge electrodes having a plurality of receptor plates arranged between the discharge electrodes. Also, other ion generating technologies can include RF, microwave, UV (or other D.C.) ion generators could be used in place of the co-axial plasma chambers in various embodiments. In other applications it will be desirable to combine different types of ion/plasma generators in the same reactor. For example, it may be desirable to combine a UV ion generator in combination with the described co-axial D.C. ion generators.

In the implementation described above with respect to FIG. 2, an ionizing electrode is grounded while a D.C. potential of as high as about 8,500 volts (or negative 8,500 volts) can be applied to the chamber walls generating current densities of on the order of 5 μA/cm² (or greater) and generate a composite electron density on the order of 10¹⁴ electrons/m³. The voltage polarity can be reversed if desired (i.e., the chamber wall grounded). At these voltage levels contaminants can be broken down into dielectric residues. For example, dimethicone (and other silicone compounds) can be broken down and enable a deposition of SiO₂ onto the discharge electrodes. These electron concentrations (and thus the ion concentration) are about two orders of magnitude higher than more conventional ion enhanced electrostatic filters. Higher potential differences can even further increase the intensity of the plasma that is generated but can also result in high incidence of electrode contamination.

### Self-Cleaning Electrodes

As mentioned above, these voltage densities can lead to extensive electrode contamination. Thus, pre-filters can be employed to reduce the amount of silicone-based materials in the air-flow. This can be supplemented by the addition of a self-cleaning electrode arrangement. In another approach the self-cleaning electrode and optionally be employed without using a carbon-based pre-filter. The following discussion describes a few example embodiments of self-cleaning discharge electrodes constructed in accordance with the principles of the invention.

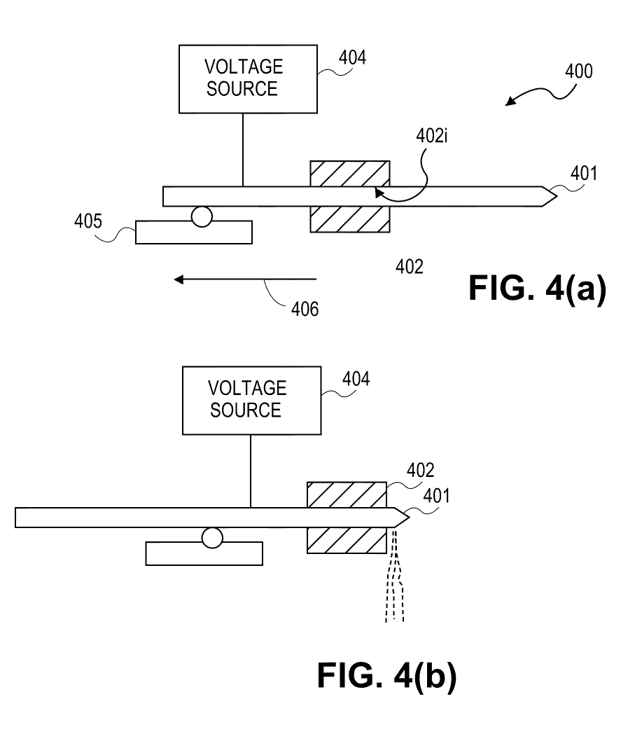

FIG. 4(a) is a cross-section view of a self-cleaning discharge electrode 400 constructed in accordance with an embodiment of the invention. The electrode 400 includes a discharge electrode shaft 401 arranged so that it can slide back and forth within a cleaning collar or sheath 402. When the electrode shaft 401 is extended into the operating position it is electrically connected with a voltage source 404 set at the desired voltage level. The electrode shaft 401 is also arranged in operative combination with an electrode retraction mechanism 405. The electrode retraction mechanism 405 moves the electrode shaft 401 from the operating position as (depicted in FIG. 4(a)) through the sheathe 402 to a retracted position (depicted in FIG. 4(b)) to enable cleaning of the shaft. Many implementations of the electrode retraction mechanism 405 can be employed to retract the shaft 401. For example, a wheel can be turned to slide the shaft 401 through the sheathe 402. Alternatively, a solenoid can be used or a magnetically actuated device and many other approaches. The invention is not limited to any particular retraction mechanism.

The inventors point out that a number or related approaches can also be employed to clean the electrode. For example, in another self cleaning approach, the discharge electrode shaft 401 remains stationary and the mechanism 405 moves the sheath 402 down the length of the shaft to enable cleaning of the shaft 401. This approach has the advantage of enabling a stationary electrical connection between the voltage source 404 and the electrode 401. In another brief example, spring-loaded actuators can be used to move the cleaning collar. Many other alternative embodiments can be employed.

In the depicted embodiment, the cleaning sheath or collar 402 includes an opening or aperture sized to match the cross-sectional dimensions of the shaft 401. During cleaning the electrode shaft 401 passes through the aperture of the cleaning sheath 402. In this embodiment, the inside diameter 402i of the aperture is sized to enable the shaft 401 to slide through with a very narrow clearance, enabling the sheath 402 to scrape off residue from the outside of the shaft 401 as it passes through the sheath 402. Also, in this embodiment, the shaft 401 has a circular cross-section that is matched by a circular aperture in the sheath 402. The inventors point out that the invention is not limited to electrodes and apertures having circular cross sections and that any suitable shape can be employed.

The sheath can be formed of dielectric or insulating materials to insulate the shaft if desired. It can also be constructed of moderately abrasive materials or other materials configured to enhance the ability of the sheath to remove unwanted residue from the shaft 401. Some suitable materials include, but are not limited to plastics and polymers (e.g., polyesters, polyethylenes, polycarbonates, polyimides, and many others), Teflon®, and hard polymers (e.g., Dyneema®, Kevlar®, and so on) can also be used. Additionally, the electrode retraction mechanism can include a motor or other operatively connected motive device (magnetic actuator, mechanical actuators, electromagnetic devices, and many others) that enables the mechanism 405 to move the shaft 401 (or alternatively, move the sheath 402) through the cleaning surfaces 402i of the sheath 402. For example, in the depicted embodiment the shaft 401 can be moved through the sheath 402 to the right (depicted by arrow 406) to enable the easy cleaning of the residue from the shaft 401. The cleaning surfaces 402i clean the shaft 401 as it passes through the sheath 402. FIG. 4(b) depicts and example electrode shaft 401 in the retracted position.

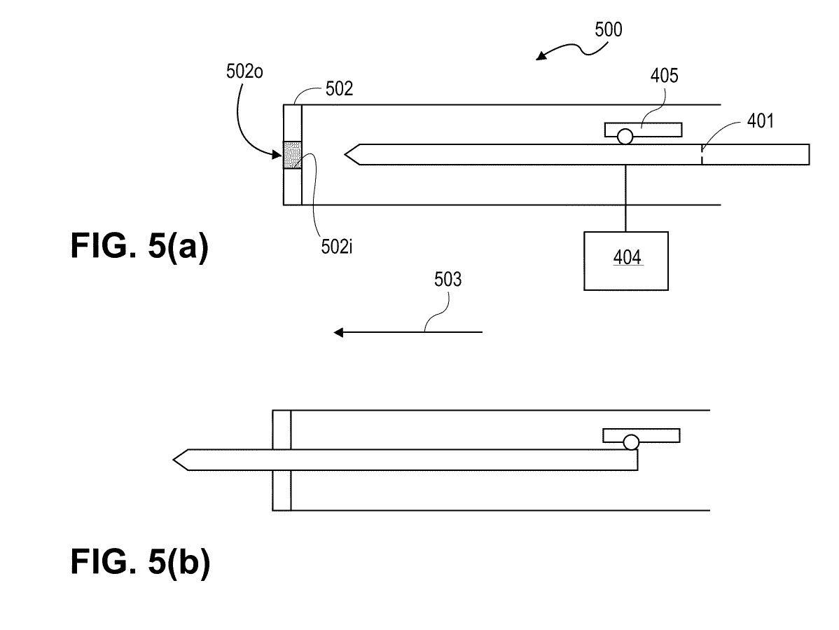

In another approach, the shaft 401 can be extended to pass through an opening in a receptor. FIG. 5(a) is a cross-section view of a self-cleaning electrode 500 constructed in accordance with an embodiment of the invention. The electrode 500 includes an electrode shaft 401 arranged so that it can slide back and forth. In order to effectuate cleaning, the shaft 401 can be extended forward until it slides through an opening 502o within a receptor electrode 502 arranged downstream from the electrode 401. When the electrode shaft 401 is extended forward from its operating position (as indicated by arrow 503) the electrode shaft 401 passes through an opening 502o in the receptor electrode 502. The inside walls 502i of the opening 502o in the receptor electrode 502 provide the cleaning surface. The opening 502i is sized to enable the shaft 401 to slide through with a very narrow clearance, enabling the walls 502i of the opening 502o to scrape off residue from the outside of the shaft 401 as it passes through the opening 502o. The receptor is commonly formed of dielectric material which electrically insulates if from the shaft if desired. Alternatively, the a separate cleaning fixture can be placed in front of the shaft 401 but up stream from the receptor 502 to enable cleaning of the shaft 401 without contact with the receptor. As before, an electrode retraction/extension mechanism 405 can be employed to move the electrode shaft 401 from its operating position to a cleaning position where the shaft slides forward (503) to engage the opening 502o to clean the residue from the shaft 401. Many possible embodiments of the retraction/extension mechanism 405 can be employed. For example, in one embodiment, the retraction/extension mechanism 405 can include a motor or other operatively connected motive device that operates to move the shaft 401 through the cleaning surfaces 502i of the opening 502o. The inventors contemplate a wide range of other motive elements including, but not limited to, magnetic actuators, mechanical actuators, spring mechanisms and many others.

FIG. 5(b) shows this embodiment as the shaft 401 is moved through the opening 502o in the direction indicated by arrow 503 to enable the easy cleaning of the residue from the shaft 401. Contact with the cleaning surfaces 502i clean the shaft 401 as it passes through the opening 502o. Additionally, in the operating position (such as depicted in FIG. 5(a)) the shaft is electrically connected with a voltage source 404 set at the desired voltage level. After cleaning the shaft is retracted back to its original operating position.

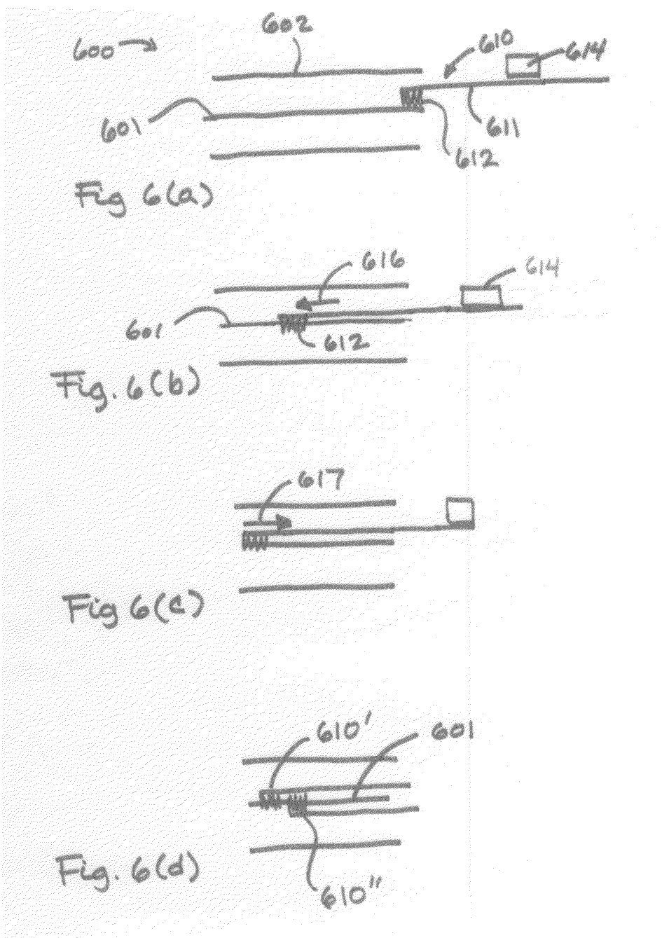

In another implementation, FIGS. 6(a)-6(d) show schematic and figurative depictions of an alternative self-cleaning discharge electrode configuration. Air flow is directed through plasma chamber 600 to enable air flow cleaning. In this cross section view, the discharge electrode 601 passes through the receptor 602. Additionally, a cleaning brush 610 including a shaft 611 having cleaning bristles 612 (not unlike those of a tooth brush) is located proximal to the electrode 601. The bristles 612 can be in contact (as shown) or not in contact with the electrode 601. In one embodiment, the bristles can be formed of polycarbonate or another stiff polymer material. Alternatively, softer or stiffer materials can be employed. Moreover, any of the materials suggested above are also applicable to this embodiment. In preferred embodiments, the bristles 612 are formed of non-conductive materials. However, the inventors contemplate a wide range of bristle materials including metals. The brush 610 can be moved back and forth by actuator 614. Such actuators can be of any suitable type, including, but not limited to those described above (e.g., the retraction mechanisms) with respect to other embodiments as well as other actuator elements.

In use (See, FIGS. 6(b)-6(c)) the actuator 610 pushes the brush 610 in one direction 616 so that the bristles 612 clean the electrode 601 as it advances along its length. The direction of the brush 610 is reversed 617 and the brush is moved backward until the electrode 601 is cleaned. In another approach described in FIG. 6(d), brushes 610′, 610″ can be arranged so that bristles are on opposite sides of the discharge electrode 601 for possibly improved cleaning. The brushes can be aligned or (as shown here) offset. In another associated approach, the cleaning apparatus can be arranges so that a cleaning brush 610 is rotated axially around the circumference of the shaft 601, thus “scrubbing” all surfaces of the shaft 601.

This method of electrode cleaning can be supplemented with other methods of electrode charge neutralization and other methods used to mitigate the effects of residue and charge build up on the electrodes.

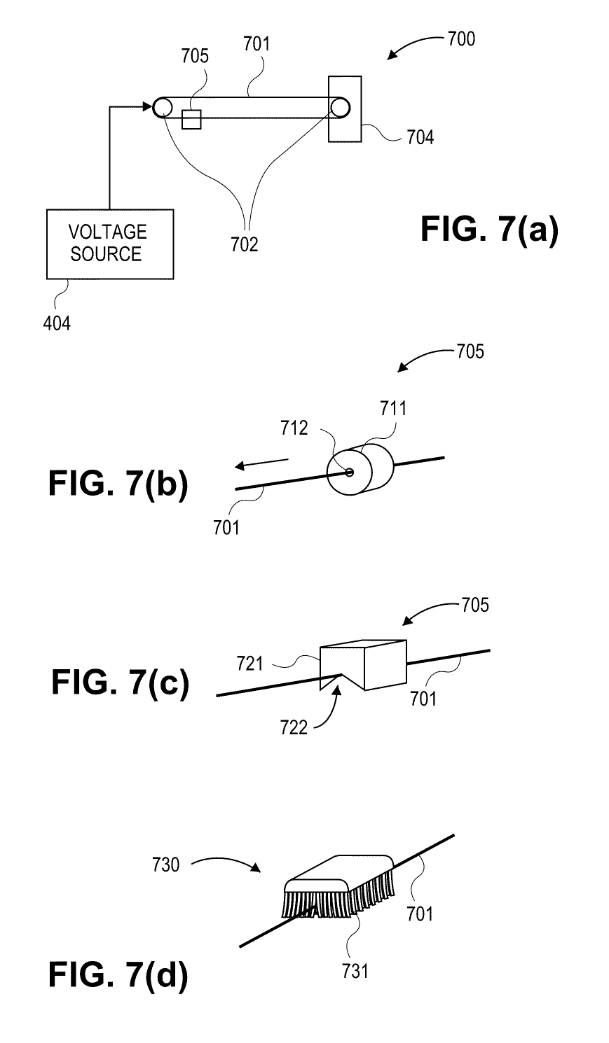

In another implementation, a “loop electrode” is employed. In such an embodiment, the discharge electrode, instead of comprising a shaft, can comprise one or more continuous strands (“loops”) of conductive material (e.g., wire) supported by two endpoints and arranged so that airflow can be passed through a reaction or ionization chamber containing the loop. In some embodiments, the loop can be further supplemented by a cleaning apparatus. The cleaning apparatus is generally configured to physically contact a cleaning surface to enable cleaning of the loop electrode. In the embodiment depicted in FIG. 7(a) a self-cleaning electrode 700 comprises a loop of conductive material 701 (e.g., wire) suspended between a pair of pulley's 702. The loop 701 is electrically connected to a voltage source 404 such as indicated elsewhere in this patent. An electrode moving mechanism 704 is arranged to enable the loop 701 to be moved over the pulleys 702. Such can be accomplished, for example, by employing a suitably constructed electrical motor 704. The inventors point out that many other modes of rotating the loop about the end pulleys can be used. Additionally, the inventors point out that the end points 704 need not be pulleys. Any surface enabling the loop 701 to rotate about the endpoints to effectuate cleaning is sufficient. For example, a set of Teflon® endpoints could be employed instead of pulleys. A cleaning element 705 is arranged to enable cleaning of the electrode loop 701 as it is moved. The electrode loop 701, or a portion thereof, is arranged in proximity with a receptor (counter-electrode) to enable ionization of the entering airflow. For example, the electrode loop 701 can be arranged inside a receptor cylinder (e.g., like FIGS. 3(a) & 3(b)) in a manner similar to that of a needle electrode. Many other approaches are also possible. A few such examples will be discussed later in this document.

FIG. 7(b) provides one example of an embodiment of a suitable cleaning element 705. The depicted cleaning element 705 comprises a sheath element 711 that has an aperture 712 arranged so that the strand of the loop 701 passes through the aperture. Accordingly, the strand is arranged in contact with the sheath 711 to enable cleaning. As the electrode moving mechanism 704 moves the loop 701 over the pulleys 702, the strand that passes through the aperture 712 rubs against the cleaning surface inside the aperture to remove residue from the loop. Rotating the loop through one cycle should clean the entire loop. Moreover, rotating through several cycles can effectuate an improved cleaning of the loop. The inventors point out that many materials are suitable for use in a cleaning element 705. Although the inventors point out that many different materials can be used, electrically insulating and dielectric materials provide excellent cleaning elements as do all of the previously discussed materials. In one embodiment, the cleaning element 705 is formed of a felt material which is abrasive enough to effectuate cleaning, yet not destructive to the loop, while still providing a sufficient level of electrical insulation.

FIG. 7(c) depicts another example of a suitable cleaning element 705. The depicted cleaning element 705 comprises a cleaning block 711 including a notch 722 (or other suitable cleaning feature) that enables cleaning of the loop 701 passes over the cleaning block 721. Thus, a block or pad 721 is drawn over a portion of the loop strand 701 (or alternatively a portion of the loop 701 is drawn over the pad). In one embodiment, the pad 721 has a notch arranged to assist in cleaning and positioning the strand. As the electrode moving mechanism 704 moves the loop 701 over the pulleys 702, the strand that passes through the notch 722 which holds the strand in place as it rubs against the surface of block to remove residue from the loop. As before, the loop can be rotated through one or many cycles to clean the loop. As explained before, many suitable materials can be employed to clean the loop. The pads may be formed using metal, dielectric materials, abrasive materials, felts, and many other suitable materials can be used in this implementation.

FIG. 7(d) depicts another example of a suitable cleaning element 730. The depicted cleaning element 730 is essentially a brush element including a set of bristles 731 arranged to enable cleaning of the loop 701 passes over the bristles 731. As the electrode moving mechanism 704 moves the loop 701 over the pulleys 702, the bristles pass over and rub against the moving loop 701 to remove residue from the loop. As before, the loop can be rotated through one or many cycles to clean the loop. As explained before, many suitable materials can be employed to in the bristles.

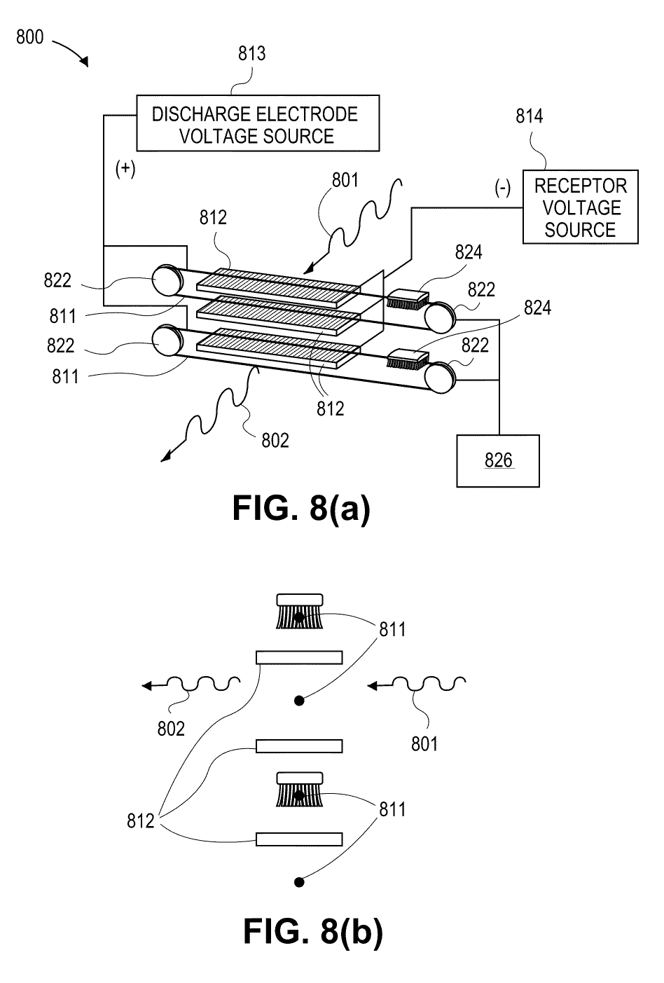

FIG. 8(a) describes another ionizer embodiment suitable for use in a plasma generator of the present invention. A series of conductive loops (such as described in FIG. 7) are arranged proximate to associated receptor electrodes in a fluid stream to enable ionization of the particulates in the air stream as it passes through the plasma generator. Such a configuration can be generally similar to that disclosed, for example, in FIG. 2. In such a configuration the plasma generator 124 includes an electrode of a type depicted in FIG. 8(a).

In such a plasma generator an upstream airflow 801 is directed through the plasma generator 800 to enable ionization of particulates in the air flow. The ionization electrodes 811 are arranged in proximity to the receptor electrodes 812 such that the arrangement enables ionization of airborne particulate passing through the plasma generator. The ionized particulates flow downstream into the electrostatic filter enabling cleaning of the particulates from the air stream and then exiting as a cleaned downstream flow 802. The plasma generator 800 configuration includes a plurality of loop ionization electrodes 811 arranged in suitable proximity to a set of receptor electrodes. This arrangement enables the formation of an ionization field between the ionization (discharge) electrodes 811 and the associated receptor electrodes 812. As the airflow passes through the ionization field the particulate in the flow become ionized. In the depicted embodiment, the discharge electrodes 811 are configured as a series conductive loops suspended on a series of associated end points 822 (e.g., pulley's) that hold the loops 811 in place and also allow them to be moved through associated cleaning elements 824 by an electrode motive element 826. Additionally, discharge electrode voltage is applied to the loops 811 by one or more voltage sources 813. Additionally, receptor electrode voltage is applied to the receptor plates 812 by one or more receptor voltage sources 814. Of course the polarity of the receptor voltage (−) is opposite from the polarity of the discharge electrode voltage (+). Also, in other embodiments, the polarity can be reversed.

Another view of this embodiment is shown in FIG. 8(b). In this depiction, the arrangement of the discharge electrodes 811 and the receptors 812 is shown. Additionally, the airflow path of unfiltered air 801 is shown, as is the outflow of ionized air 802.

One or more cleaning elements 824 are moved over the electrodes 811 in order to remove accumulated residue from the loops. Typically, the movement is accomplished by one or more associated electrode motive elements (abstractly depicted as element 826) are activated to move the loops 811. As described in detail above, as the loops are rotated over the pulleys 822 they are cleaned by elements 824 thereby cleaning the loops. Some example cleaning elements are described above, for example, with respect to FIGS. 7(a)-7(d).

This method of cleaning can be supplemented with other methods of grid charge neutralization and other methods used to mitigate the effects of residue and charge build up on the grids.

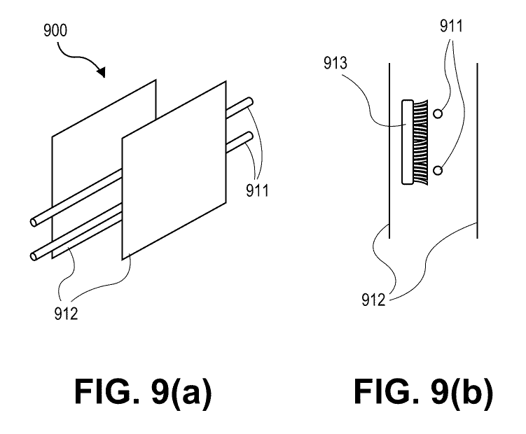

FIGS. 9(a)-9(b) depict another self cleaning electrode embodiment. In this plasma generator embodiment 900, a pair of ionization electrodes 911 are arranged between a pair of receptor electrodes 912 such that an ionization field is created between the plates and ionization electrodes. Additionally, as depicted in FIG. 9(b) a cleaning element 913 is arranged so that it can be contacted against the electrodes 911 as it moves between the plates 912 along the length of the electrodes 911. The depicted embodiment uses a brush cleaning element 913 although others can be used. The movement of the cleaning element 913 can be facilitated by a number of motive elements. For example, it can be pushed along using a mechanism like that described in FIG. 6. Also, the cleaning element 913 can be suspended between a pair of wires and moved back and forth over the electrode by the wires. Also, alternatively, the cleaning element 913 and electrodes 911 can be moved to contact each other and then the electrodes can be moved across the cleaning surface of the cleaning element 913. The inventors also contemplate that the cleaning element 913 in this embodiment (and other embodiments) can be used to clean the receptors.

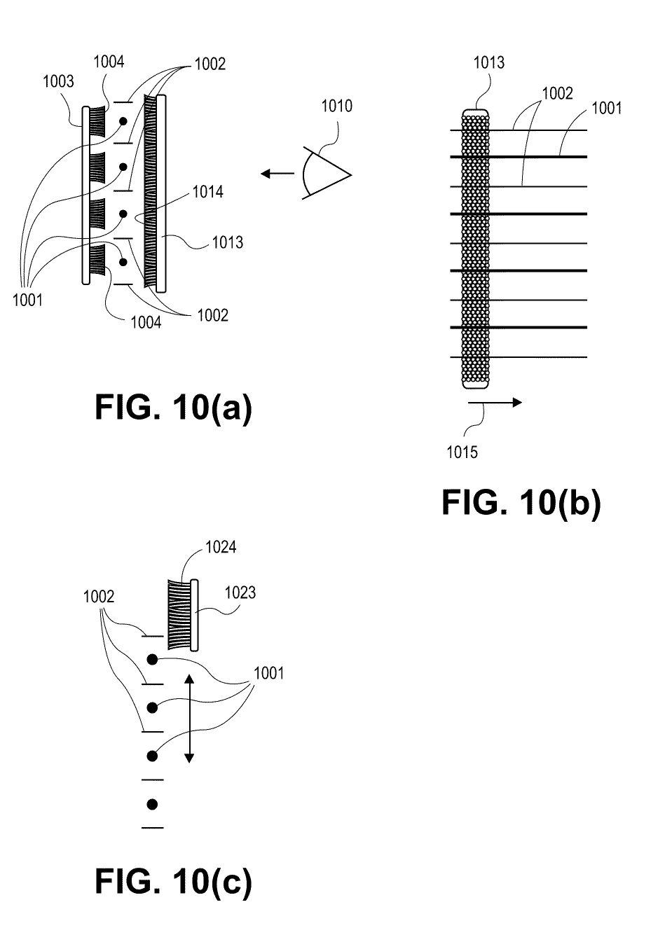

FIG. 10(a) provides a depiction of another embodiment suitable for use in a plasma generator of the present invention. In this embodiment a plurality of discharge electrodes 1001 are arranged between a plurality of receptor electrodes 1002 to enable ionization of the particulates in the air stream as they pass through the plasma generator. It is pointed out that in FIG. 10(a) the electrodes 1001, 1002 are elongate extending into the page similar to the depiction of FIG. 8(b). A single cleaning element 1003 extends across the electrodes as shown in FIG. 10(a). Cleaning surfaces 1004 are contacted to the discharge electrodes 1001. The cleaning element 1003 is passed into or out of the page to effectuate cleaning of the discharge electrodes 1001.

In a related approach, FIG. 10(a) also provides a depiction of another self-cleaning embodiment suitable for use in a plasma generator of the present invention. In this embodiment a cleaning element 1013 extends across the electrodes as shown. A cleaning surface 1014 extends contiguously across the electrodes 1001, 1002. The surface 1014 is contacted to both the discharge and the receptor (or counter electrodes) electrodes 1001. The cleaning element 1003 is passed into or out of the page to effectuate cleaning of the discharge and receptor electrodes 1001, 1002.

FIG. 10(b) shows a view of the embodiment of FIG. 10(a) as viewed from 1010. This view shows clearly, a path 1015 that a cleaning element 1013 can move across the electrodes 1001, 1002 is shown. The cleaning surface 1014 extends contiguously across the electrodes 1001, 1002. As stated above, as the cleaning surface 1014 is contacted to both the discharge and the receptor electrodes 1001, 1002 and moved across the electrodes all the electrodes are cleaned at once.

In another embodiment, FIG. 10(c) shows another view similar to that of FIG. 10(a). In this embodiment, a cleaning element 1023 is moved across the electrodes 1001, 1002 is shown. The cleaning surface 1024 extends contacts across the electrodes 1001, 1002. As the cleaning surface 1024 is contacted to both the discharge and the receptor electrodes 1001, 1002 and moved across the electrodes all the electrodes are cleaned.

In the foregoing descriptions, the plasma generators and the various self-cleaning electrodes have been described as having potentials applied thereto. These plasma generators can be sealed and still enable the self-cleaning electrodes to function without needing to open the devices or actively service the devices. Therefore, the present embodiments are to be considered as illustrative and not restrictive and the invention is not to be limited to the details given herein, but may be modified within the scope and equivalents of the appended claims.

1. A plasma reactor comprising:
a plasma chamber having a discharge electrode and a receptor arranged to form an ionization field through which an incoming airflow is passed; and
a cleaning element arranged in the plasma chamber to clean at least the discharge electrode.

2. The plasma reactor of claim 1 wherein a carbon-containing pre-filter element is arranged to filter the incoming airflow before passing it into the plasma chamber.

3. The plasma reactor of claim 2 wherein a dielectric enhanced electrostatic filter element is arranged to filter air flow after plasma treatment and carbon filtering.

4. The plasma reactor of claim 1 wherein a pre-filter element is arranged to filter silicone containing materials from an incoming airflow before it into the plasma chamber.

5. The plasma reactor of claim 4 wherein the pre-filter element comprises a non-carbon filter element arranged to receive an incoming airflow and a carbon filter element arranged between the non-carbon filter element and the plasma chamber to receive filtered airflow from the non-carbon filter element for filtration and exhausting into the plasma chamber.

6. The plasma reactor of claim 1 wherein the discharge electrode comprises at least one continuous strand of material, each strand supported by a pair of endpoints that enable the strand to be rotated about the endpoints.

7. The plasma reactor of claim 6 wherein said receptor includes at least one receptor element with at least one receptor element arranged between portions of a strand of said discharge electrode.

8. The plasma reactor of claim 1 wherein the self-cleaning electrode comprises:
a cleaning element comprising at least one brush element arranged in proximity to the discharge electrode; and
a motive mechanism engaged with the brush element to enable the brush to be moved along a surface of the discharge electrode cleaning the electrode.

9. A plasma reactor comprising:
a carbon-containing pre-filter element;
a plasma chamber having a discharge electrode and a receptor arranged to form an ionization field through which inflowing air flows; and
a dielectric enhanced electrostatic filter element arranged to filter air flow after plasma treatment.

10. A self-cleaning electrode for use in a plasma reactor, the self-cleaning electrode comprising:
a cleaning element comprising a collar with an aperture formed therein, the aperture sized to such that an inner diameter of the aperture sized to create cleaning contact with an electrode shaft of a plasma reactor;
the electrode shaft having an elongate dimension and cross-sectional dimension, the shaft arranged so that it extends through the aperture of the cleaning element; and
a motive mechanism enables the shaft to be moved relative to the collar enabling the shaft to slide through the aperture cleaning the shaft.

11. The self-cleaning electrode of claim 10 further configured such that a cross-sectional dimension of the electrode is closely matched by the inner diameter of the aperture of the collar enabling contact between the collar and the shaft to clean the shaft.

12. The self-cleaning electrode of claim 11 further configured such that an inner surface of the aperture of the collar is abrasive enhancing its cleaning effect by physical contact with an outer surface of the electrode shaft.

13. The self-cleaning electrode of claim 11 further configured such that at least an inner surface of the aperture of the collar is formed of a dielectric material.

14. The self-cleaning electrode of claim 10 further including a voltage source in electrical contact with the electrode shaft.

15. The self-cleaning electrode of claim 10 wherein the motive mechanism moves the shaft through the aperture while the collar remains stationary.

16. The self-cleaning electrode of claim 10 wherein the motive mechanism moves the collar such that the shaft passes through the aperture while the shaft remains stationary.

17. A self-cleaning electrode for use in a plasma reactor, the self-cleaning electrode comprising:
a cleaning element;
a continuous strand of conductive material looped between two end points to form an electrode, wherein a portion of the strand is in physical contact with the cleaning element; and
an electrode moving mechanism that enables the continuous strand of conductive material to be moved about the two endpoints and maintain contact with the cleaning element while the loop moves thereby cleaning the loop as it moves.

18. The self-cleaning electrode of claim 17 wherein,
the cleaning element is configured to include an aperture, and
the strand of conductive material is arranged such that the strand passes through the aperture.

19. The self-cleaning electrode of claim 18 wherein the two endpoints comprise pulleys.

20. The self-cleaning electrode of claim 19 further including a voltage source in electrical contact with the strand of conductive material.

21. The self-cleaning electrode of claim 17 wherein,
the cleaning element is includes a notch formed therein, and
the strand of conductive material is arranged such that the strand contacts the cleaning element in the notch.

22. A plasma reactor for collecting aerosol particulates carried in a fluid stream passing through the reactor, the plasma reactor comprising:
a prefilter that receives a fluid stream and filters at least some particulates from the fluid stream;
at least one plasma chamber located downstream from the prefilter and configured to include a self-cleaning electrode for cleaning the electrode to remove residue from the electrode, the plasma chamber configured such that the prefiltered stream is treated by the electrode to increase the concentration of reactive species in the stream;
at least one electrostatic filter located downstream from the plasma chamber for electrostatically filtering particles from the fluid stream; and
a catalyst located downstream from the electrostatic filter and arranged to reduce the concentration of reactive species that are contained in the fluid stream before the fluid stream emerges from the plasma reactor.

23. The plasma reactor recited in claim 22 wherein the self-cleaning electrode comprises:
a cleaning element having an aperture formed therein, the aperture sized to match a cross-sectional dimension of an electrode shaft;
an electrode shaft having an elongate dimension and cross-sectional dimension, the shaft arranged so that the elongate dimension of the shaft extends coaxially down a length of the plasma chamber and also extends through the aperture of the cleaning element; and
a shaft retraction mechanism that enables to shaft to be moved through the aperture to enable the shaft to be cleaned by contact with inside surfaces of the aperture.

24. The plasma reactor recited in claim 22 wherein the self-cleaning electrode comprises:
a continuous strand of conductive material suspended between two end points;
a cleaning element arranged so that a strand is in physical contact with the cleaning element; and
an electrode moving mechanism that enables the strand of conductive material to be moved about the two endpoints and maintain contact with the cleaning element while the strand moves thereby cleaning the strand as it moves.

25. A plasma reactor as recited in claim 22 wherein the plasma in the plasma chamber is created using at least one of an RF and a microwave plasma generator.

26. A plasma reactor as recited in claim 22 further comprising a UV ion generator.

27. A plasma reactor as recited in claim 22 wherein the pre-filter includes a carbon filter element.

28. A plasma reactor comprising:
a pre-filter element configured to filter silicone-based contaminants from an incoming airstream;
a plasma chamber arranged to receive the inflowing airstream from the pre-filter element, the plasma chamber having a discharge electrode and a receptor arranged to form an ionization field through which airstream flows; and
a dielectric enhanced electrostatic filter element arranged to filter the airstream after plasma treatment.

29. The plasma reactor of claim 28 wherein the plasma chamber includes a cleaning element arranged to clean portions of the plasma chamber.

30. The plasma reactor of claim 29 wherein the cleaning element is arranged to clean portions at least one of the discharge electrode and the receptor.
